(19) United States
(12) Patent Application Publication
(10) Pub. No.: US 2012/0003319 A9
(43) Pub. Date: Jan. 5, 2012

(54) COMPOSITIONS FOR SITE-SPECIFIC DELIVERY OF IMATINIB AND METHODS OF USE

(75) Inventors: Gary Liversidge, West Chester, PA (US); Scott Jenkins, Downingtown, PA (US)

(73) Assignee: ELAN PHARMA INTERNATIONAL LIMITED, County Westmeath (IE)

(21) Appl. No.: 12/407,684

(22) Filed: Mar. 19, 2009

Related U.S. Application Data
(63) Continuation-in-part of application No. 12/071,849, filed on Feb. 27, 2008.
Application No. 12/071,849 is a continuation of application No. 11/300,592, filed on Dec. 15, 2005, now abandoned.
(60) Provisional application No. 60/731,869, filed on Nov. 1, 2005.
(60) Provisional application No. 60/636,817, filed on Dec. 15, 2004.

(51) Int. Cl. A61K 9/14; A61K 31/497

(52) U.S. Cl. 424/490; 514/252.18

(57) ABSTRACT

The invention provides an oral formulation for administering to a subject comprising an imatinib compound and an enteric matrix or enteric coating or a combination thereof, whereby at least 80% of the imatinib compound is released in the small intestine of the subject. Methods of using such formulation is also provided.

### RELATIONSHIP TO PRIOR APPLICATIONS

The instant application claims priority under 35 U.S.C. §119(e) to U.S. Provisional Application 61/038,524, filed on Mar. 21, 2008, and to U.S. Provisional Application 61/038,892, filed on Mar. 24, 2008. Each of these applications is incorporated herein by reference in its entirety.

### FIELD OF THE INVENTION

This invention is in the field of formulations comprising imatinib, and methods of using such formulations.

### BACKGROUND

Imatinib is a protein tyrosine kinase inhibitor that inhibits the bcr-abl tyrosine kinase, the constitutive abnormal tyrosine kinase created by the Philadelphia chromosome abnormality in chronic myeloid leukemia (CML). Imatinib induces proliferation and induces apoptosis in bcr-abl positive cell lines as well as fresh leukemic cells from Philadelphia chromosome positive myeloid leukemia. In colony formation assays using ex vivo peripheral blood and bone marrow samples, imatinib shows inhibition of bcr-abl positive colonies from CML patients.

In vivo, imatinib inhibits tumor growth of bcr-abl transfected murine myeloid cells as well as bcr-abl positive leukemia lines derived from CML patients in blast crisis. Imatinib is also an inhibitor of the receptor tyrosine kinases for platelet-derived growth factor (PDGF) and stem cell factor (SCF) and c-kit, and it inhibits PDGF- and SCF-mediated cellular events. In vitro, imatinib inhibits proliferation and induces apoptosis in gastrointestinal stromal tumor (GIST) cells, which express an activating c-kit mutation.

Imatinib is administered to patients in form of imatinib mesylate. Imatinib mesylate is a white to off-white to brownish or yellowish tinged crystalline powder. Imatinib mesylate is chemically known as 4-[(4-Methyl-1-piperazinyl)methyl]-N-[4-methyl-3-[[4-(3-pyridinyl)-2-pyrimidinyl]amino]-phenyl]benzamide methanesulfonate. Its molecular formula is C₂₉H₃₁N₇O.CH₄SO₃, and its molecular weight is 589.7. The structure of imatinib mesylate is shown in Formula I below:

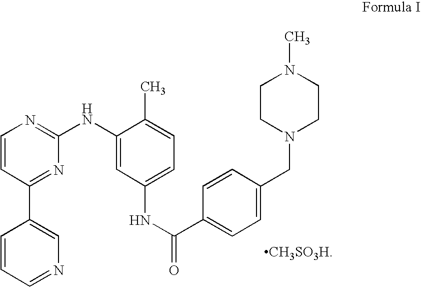

Imatinib mesylate is very soluble in water and soluble in aqueous buffers≦pH 5.5 but is very slightly soluble to insoluble in neutral/alkaline aqueous buffers. In non-aqueous solvents, the drug substance is freely soluble to very slightly soluble in dimethyl sulfoxide, methanol and ethanol, but is insoluble in n-octanol, acetone and acetonitrile. Imatinib mesylate compounds have been disclosed, for example, in U.S. Pat. No. 5,521,184 to Zimmermann for “Pyrimidine Derivatives and Processes for the Preparation Thereof” and United States Patent Application No. Publication 2004/0127571 to Bhalla et al. for “Method of Treating Leukemia with a Combination of Suberoylanilide Hydromaxic Acid and Imatinib Mesylate”. Both of these references are hereby incorporated by reference.

Imatinib mesylate is sold under brand name Gleevec®. Gleevec® film-coated tablets contain imatinib mesylate equivalent to 100 mg or 400 mg of imatinib free base. Gleevec® also includes the following inactive ingredients: colloidal silicon dioxide (NF), crospovidone (NF), magnesium stearate (NF) and microcrystalline cellulose (NF). The tablets are coated with ferric oxide, red (NF); ferric oxide, yellow (NF); hydroxyproply methylcellulose (USP); polyethylene glycol (NF) and talc (USP).

Gleevec® is generally prescribed in dosages of 400 mg/day for adult patients in chronic phase CML and 600 mg/day for adult patients in accelerated phase or blast crisis. Additionally, Gleevec® is recommended at dosages of 400 mg/day or 600 mg/day for adult patients with unresectable and/or metastatic, malignant GIST. Gleevec® is generally prescribed to be administered orally, with a meal and a large glass of water, with doses of 400 mg or 600 mg administered once daily, and dosages of 800 mg administered as 400 mg twice a day.

Intake of imatinib, however, is associated with undesirable side effects, including, without limitation, edema, nausea, vomiting, fatigue, muscle cramps, diarrhea, abdominal pain, and other adverse reactions.

Accordingly, there is a need for improved imatinib formulations which do not affect the effectiveness of imatinib while decreasing or eliminating at least some of its side effects.

### SUMMARY OF INVENTION

The inventors have observed that IV administration of imatinib eliminates the incidence of emesis and concluded that it is likely that emesis results from local gastric effect of imatinib. The severity and/or frequency of this unwanted side effect can therefore be diminished or altogether eliminated if imatinib is administered in a formulation which prevents or decreases imatinib release in the stomach of the subject. Additionally, other upper GI side effects such as dyspepsia will also be prevented or decreased by releasing imatinib in the intestine.

Accordingly, the instant invention addresses the drawbacks of the current imatinib formulations by providing, in one aspect, an oral formulation for administering to a subject containing an imatinib compound and an enteric matrix or enteric coating or a combination thereof; whereby at least 80% of the imatinib compound is released in the small intestine of the subject.

In one set of embodiments, at least a portion of the imatinib compound of the oral formulation is in a nanoparticulate form, and the nanoparticles of the imatinib compound further comprise at least one surface stabilizer. In some embodiments, the formulation comprises at least a second active ingredient, which may optionally be present in nanoparticulate form. In some embodiments, at least the second active ingredient is selected from anti-emetic compounds, anti-diarrhea compounds, and H₂ antagonists.

In another aspect, the invention provides a method of method of treating a subject having a disease amenable to imatinib therapy, comprising administering to a subject a formulation according to any embodiment of the previous aspect of the invention. In one embodiment, the method administers a single daily dose of the formulation having the equivalent of about 800 mg of imatinib.

### DETAILED DESCRIPTION

For the purpose of a better understanding the instant application, the following definitions are provided:

The term “about” will be understood by persons of ordinary skill in the art and will vary to some extent on the context in which it is used. If there are uses of the term which are not clear to persons of ordinary skill in the art given the context in which it is used, “about” will mean up to plus or minus 10% of the particular term.

The phrase “poorly soluble drug” refers to those drugs that are poorly soluble in aqueous media such as water, at neutral pH. For example, poorly soluble drugs are those drugs with a solubility in aqueous media, at neutral pH, of less than about 30 mg/ml, less than about 20 mg/ml, less than about 10 mg/ml, or less than about 1 mg/ml.

Aqueous solubility may be determined by any appropriate method known in the art. For example, solubility may be determined by adding the therapeutic agent to stirred or agitated medium maintained in a constant temperature bath at a temperature of 37° C. until equilibrium is established between the dissolved and undissolved states and the concentration of dissolved drug is constant. The resulting solution saturated with active agent may then be filtered, typically under pressure through a 0.8-micron Millipore filter, and the concentration in solution may be measured by any appropriate analytical method including gravimetric, ultraviolet spectrophometry, chromatography.

The term “effective average particle size of less than about 2000 nm,” as used herein, means that at least about 50% of the nanoparticulate imatinib mesylate particles have a size of less than about 2000 nm, by weight (or by other suitable measurement technique, such as by number, volume, etc.) when measured by, for example, sedimentation flow fractionation, photon correlation spectroscopy, light scattering, disk centrifugation, and other techniques known to those of skill in the art.

As used herein with reference to stable imatinib mesylate nanoparticulate particles, “stable” connotes, but is not limited to one or more of the following parameters: (1) the particles do not appreciably flocculate or agglomerate due to interparticle attractive forces or otherwise significantly increase in particle size over time; (2) that the physical structure of the particles is not altered over time, such as by conversion from an amorphous phase to a crystalline phase; (3) that the particles are chemically stable; and/or (4) where the imatinib mesylate has not been subject to a heating step at or above the melting point of the imatinib mesylate in the preparation of the nanoparticles of the present invention.

The term “conventional” or “non-nanoparticulate active agent” shall mean an active agent which is solubilized or which has an effective average particle size of greater than about 2000 nm. Nanoparticulate active agents as defined herein have an effective average particle size of less than about 2000 nm.

Generally, the invention provides a formulation comprising an imatinib compound and an enteric matrix, or enteric coating, or a combination of the enteric matrix and the enteric coating. The imatinib compound may be present in a form of a free base (i.e., imatinib per se) or as a salt of imatinib, including, without limitation, imatinib mesylate.

Derivatives of imatinib are also may be used. In one embodiment, the imatinib compound is described by Formula II below:

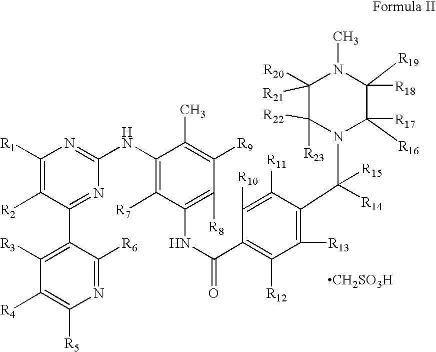

In different embodiments encompassed by Formula II, each substituent R¹-R²³, may be the same or different, and is selected, independently from each other, from a group consisting of —H; —OH; —F; —Cl; —Br; —I; —NH₂; alkyl- and dialkylamino; linear or branched C1-6 alkyl, C₂₋₆ alkenyl and alkynyl; aralkyl; linear or branched C₁₋₆ alkoxy; aryloxy; aralkoxy; -(alkylene)oxy(alkyl); —CN; —NO₂; —COOH; —COO(alkyl); —COO(aryl); —C(O)NH (C₁₋₆ alkyl); —C(O)NH(aryl); sulfonyl; (C₁₋₆ alkyl)sulfonyl; arylsulfonyl; sulfamoyl, (C₁₋₆ alkyl)sulfamoyl; (C₁₋₆ alkyl)thio; (C₁₋₆ alkyl)sulfonamide; arylsulfonamide; —NHNH₂; —NHOH; aryl; and heteroaryl; and where each alkyl, alkenyl, alkynyl, aryl, and heteroaryl moiety may be optionally substituted with one or more groups independently selected from the group consisting of —OH; —F; —Cl; —Br; —I; —NH₂; alkyl- and dialkylamino; linear or branched C₁₋₆ alkyl, C₂₋₆ alkenyl and alkynyl; aralkyl; linear or branched C₁₋₆ alkoxy, aryloxy; aralkoxy; -(alkylene)oxy(alkyl); —CN, —NO₂, —COOH, —COO(alkyl); —COO(aryl); —C(O)NH(C₁₋₆ alkyl); —C(O)NH(aryl); sulfonyl; (C₁₋₆ alkyl)sulfonyl; arylsulfonyl; sulfamoyl, (C₁₋₆ alkyl)sulfamoyl; (C₁₋₆ alkyl)thio; (C₁₋₆ alkyl)sulfonamide; arylsulfonamide; —NHNH₂; and —NHOH.

The imatinib compound is formulated as to prevent its local effect on the stomach of the patient and thus to diminish or eliminate the incidence of nausea and/or vomiting. In one embodiment, this result is achieved by coating the imatinib compound with a substrate which is poorly soluble or insoluble in gastric environment (e.g., at pH below 2.5) but soluble at higher pH, such as, e.g., from about 4 to about 8. This feature of the enteric coating ensures that at least 80% of the imatinib compound is released in the subject's small intestine. Preferably, at least about 85% the imatinib compound is released in the subject's small intestine, more preferably, about 90% the imatinib compound is released in the subject's small intestine, more preferably, about 95%, and particularly preferably, about 100% the imatinib compound is released in the subject's small intestine.

Suitable enteric coatings are well known in the art and include, without limitation, polymer coating materials, such as cellulose acetate phthalate, cellulose acetate trimaletate, hydroxy propyl methylcellulose phthalate, polyvinyl acetate phthalate, ammonio methacrylate copolymers such as those sold under the tradename EUDRAGIT® RTM, RS, and RL, poly acrylic acid and poly acrylate and methacrylate copolymers such as those sold under the tradename EUDRAGIT® S and L, polyvinyl acetaldiethylamino acetate, hydroxypropyl methylcellulose acetate succinate, shellac; hydrogels and gel-forming materials, such as carboxyvinyl polymers, sodium alginate, sodium carmellose, calcium carmellose, sodium carboxymethyl starch, poly vinyl alcohol, hydroxyethyl cellulose, methyl cellulose, gelatin, starch, and cellulose based cross-linked polymers in which the degree of crosslinking is low so as to facilitate adsorption of water and expansion of the polymer matrix, hydroxypropyl cellulose, hydroxypropyl methylcellulose, polyvinylpyrrolidone, crosslinked starch, microcrystalline cellulose, chitin, aminoacryl-methacrylate copolymer (EUDRAGIT® RS-PM, Rohm & Haas), pullulan, collagen, casein, agar, gum arabic, sodium carboxymethyl cellulose, (swellable hydrophilic polymers) poly(hydroxyalkyl methacrylate) (m. wt. about 5 k-5,000 k), polyvinylpyrrolidone (m. wt. ˜10 k-360 k), anionic and cationic hydrogels, polyvinyl alcohol having a low acetate residual, a swellable mixture of agar and carboxymethyl cellulose, copolymers of maleic anhydride and styrene, ethylene, propylene or isobutylene, pectin (m. wt. ˜30 k-300 k), polysaccharides such as agar, acacia, karaya, tragacanth, algins and guar, polyacrylamides, POLYOX®, polyethylene oxides (m. wt. ˜100 k -5,000 k), AQUAKEEP® acrylate polymers, diesters of polyglucan, crosslinked polyvinyl alcohol and poly N-vinyl-2-pyrrolidone, sodium starch glucolate (e.g. EXPLOTAM®; Edward Mandell C. Ltd.); hydrophilic polymers such as polysaccharides, methyl cellulose, sodium or calcium carboxymethyl cellulose, hydroxypropyl methyl cellulose, hydroxypropyl cellulose, hydroxyethyl cellulose, nitro cellulose, carboxymethyl cellulose, cellulose ethers, polyethylene oxides (e.g. POLYOX®, Union Carbide), methyl ethyl cellulose, ethylhydroxy ethylcellulose, cellulose acetate, cellulose butyrate, cellulose propionate, gelatin, collagen, starch, maltodextin, pullulan, polyvinyl pyrrolidone, polyvinyl alcohol, polyvinyl acetate, glycerol fatty acid esters, polyacrylamide, polyacrylic acid, copolymers of methacrylic acid or methacrylic acid (e.g. EUDRAGIT®, Rohm and Haas), other acrylic acid derivatives, sorbitan esters, natural gums, lecithins, pectin, alginates, ammonia alginate, sodium, calcium, potassium alginates, propylene glycol alginate, agar, and gums such as arabic, karaya, locust bean, tragacanth, carrageens, guar, xanthan, scleroglucan and mixtures and blends thereof.

As will be appreciated by the person skilled in the art, excipients such as plasticisers, lubricants, solvents and the like may be added to the coating. Suitable plasticisers include for example acetylated monoglycerides; butyl phthalyl butyl glycolate; dibutyl tartrate; diethyl phthalate; dimethyl phthalate; ethyl phthalyl ethyl glycolate; glycerin; propylene glycol; triacetin; citrate; tripropioin; diacetin; dibutyl phthalate; acetyl monoglyceride; polyethylene glycols; castor oil; triethyl citrate; polyhydric alcohols, glycerol, acetate esters, gylcerol triacetate, acetyl triethyl citrate, dibenzyl phthalate, dihexyl phthalate, butyl octyl phthalate, diisononyl phthalate, butyl octyl phthalate, dioctyl azelate, epoxidised tallate, triisoctyl trimellitate, diethylhexyl phthalate, di-n-octyl phthalate, di-i-octyl phthalate, di-i-decyl phthalate, di-n-undecyl phthalate, di-n-tridecyl phthalate, tri-2-ethylhexyl trimellitate, di-2-ethylhexyl adipate, di-2-ethylhexyl sebacate, di-2-ethylhexyl azelate, and dibutyl sebacate.

Further, exemplary enteric coatings contemplated by the present invention include those disclosed in the following patents, each of which is incorporated by reference.

One exemplary enteric coating composition contemplated by the present invention is disclosed by K. G. Wagner et al., Anion-induced Water Flux as Drug Release Mechanism Through Cationic Euragit RS 30D Film Coatings, The AAPS Journal 2005, 7(3) Article 67, E668-E677. Wagner discloses polymer-coating compositions for sustained release oral dosage forms using cationic polymethacrylate sold under the tradename EUDRAGIT® RD by Degussa GmbH, of Dusseldorf, DE.

Another exemplary enteric coating composition contemplated by the present invention is disclosed by N. Huyghebaert et al., In vitro Evaluation of Coating polymers for Enteric Coating and Human Ileal Targeting, International Journal of Pharmaceutics, 2898 (2005), 26-27. Huyghebaert et al. studied numerous cationic polymethacrylates sold under the tradename EUDRAGIT® for evaluation of enteric properties and ileal targeting.

Another embodiment of the present invention comprises the imatinib compound, distributed throughout a tablet matrix. With the pharmaceutically acceptable type and amount of surfactants and or excipients, the tablet, when ingested, will erode the drug in amounts sufficient to present the drug in a physiologically absorbable form.

Suitable matrix materials contemplated by the present invention include hydrophilic polymers, hydrophobic polymers and mixtures thereof, including but are not limited to, microcrytalline cellulose, sodium carboxymethylcellulose, hydoxyalkylcelluloses such as hydroxypropylmethylcellulose and hydroxypropylcellulose, polyethylene oxide, alkylcelluloses such as methylcellulose and ethylcellulose, polyethylene glycol, polyvinylpyrrolidone, cellulose acteate, cellulose acetate butyrate, cellulose acteate phthalate, cellulose acteate trimellitate, polyvinylacetate phthalate, polyalkylmethacrylates, polyvinyl acetate and mixture thereof.

One such matrix material comprises one or more excipients selected from the group of fatty alcohol, triglyceride, partial glyceride and fatty acid ester as taught in U.S. Pat. No. 7,175,854, herein incorporated by reference. According to one example, the active ingredient is dispersed i) in an excipient matrix composed of a mixture comprising at least one fatty alcohol and at least one solid paraffin, ii) in an excipient matrix comprised of a mixture comprising at least one triglyceride and at least one solid paraffin, iii) in an excipient matrix composed of a mixture comprising at least one partial glyceride and at least one solid paraffin or iv) in an excipient matrix composed of a mixture comprising at least one fatty acid ester and at least one solid paraffin. These matrices are highly stabile, release the active ingredient in a controlled manner by the particle size and composition of the matrix, exhibit good flow characteristics, good compressibility by a uniform delivery of active ingredient. In the case of acid-labile active ingredients, e.g., the imatinib compound, it is possible to achieve, through choice of the matrix excipients, an acid resistance so that it is possible in the case of oral forms to dispense with an acid-resistant coating (i.e., enteric coating).

Another suitable matrix of the present invention is described in U.S. Pat. No. 7,157,100 to Doshi et al. (“the '100 Patent), hereby incorporated by reference. The '100 Patent discloses a controlled release multilayer composition comprising a matrix forming gelling agent which is intended for controlled delivery of active agent to maintain therapeutic effective concentrations. The matrix forming gelling agents are selected from group consisting of hydroxypropyl methylcellulose, methylcellulose, hydroxypropyl cellulose, carbomer, carboxy methylcellulose, gum tragacanth, gum acacia, guar gum, pectin, modified starch derivatives, xanthan gum, locusta bean gum, sodium alginate, the most preferred being hydroxypropyl methylcellulose, i.e. Methocel®, which on contact with gastric fluid swells and gels, forming matrix structure that entraps the gas released and also release the active agent in a controlled manner.

Another matrix forming gelling agent of the '100 Patent is hydroxypropyl methylcellulose which has a viscosity in the range from 4,000 cps to about 100,000 cps. Suitable commercially available hydroxypropyl methylcellulose (viscosity 3000 5600 cP) is available under the trademark Methocel® K4M and methyl cellulose (viscosity 80000 120000 cP) available under the trademark Methocel® K100M.

Another suitable matrix composition contemplated by the present invention includes those described in M. Baluom, et al., Synchronized Release of Sulpiride and Sodium Decanoate from HPMC Matrices: A Rational Approach to Enhance Sulpiride Absorption in the Rat Intestine, Pharmaceutical Research, Vol 17, No. 9, (2000) 1071-1076, herein incorporated by reference. Baluom et al. disclose matrix compositions comprising varying amounts of sodium decanoate and HPMC and their different erosion rates. Yet a further matrix composition contemplated by the present invention is disclosed in M. H. Amaral, et al., Effect of Hydroxypropyl Methylcellulose and Hydrogenated Caster Oil in Naproxene Release From Sustained-Release Tablets, AAPS PharmSciTech 2001; 2 (2) article 6 and R. O. Williams III, et al., Method to Recover a Lipophilic Drug from Hydroxypropyl Methylcellulose Matrix Tablets, AAPS PhramSciTech 2001, 2 (2) article 8, both of which are incorporated by reference herein. Amaral, et al. discloses the effect of varying compositions of double compressed matrix tablets comprising hydrophilic (HPMC) and hydrophobic (hydrogenated caster oil) products, filler, and buffers on the release rate of naproxene in rats.

Still further suitable dispersion compositions contemplated by the present invention includes those compositions disclosed in U.S. Publications 20060177500 and its corresponding PCT publication WO 2005004848 both of which have the title “Solid Dispersion of Tacrolimus”; and K. Yamashita, et al., establishment of New Preparation Method for Solid Dispersion Formulation of Tacrolimus, International journal of Pharmaceutics 267 (2003) 79-91, all of which are incorporated by reference herein.

In yet another embodiment, the imatinib compound may be in a form of an emulsion or suspension, encapsulated within the enteric coating. Exemplary emulsifiers include, without limitation, ethyl alcohol, isopropyl alcohol, ethyl carbonate, ethyl acetate, benzyl alcohol, benzyl benzoate, propyleneglycol, 1,3-butyleneglycol, dimethylformamide, oils, such as cottonseed oil, groundnut oil, corn germ oil, olive oil, castor oil, and sesame oil, glycerol, tetrahydrofurfuryl alcohol, polyethyleneglycols, fatty acid esters of sorbitan, or mixtures of these substances, and the like.

Additional non-limiting examples of controlled release matrices are described in U.S. Pat. Nos. 6,326,027; 6,340,475; 6,905,709; 6,645,527; 6,576,260; 6,326,027; 6,254,887; 6,306,438; 6,129,933; 5,891,471; 5,849,240; 5,965,163; 6,162,467; 5,567,439; 5,552,159; 5,510,114; 5,476,528; 5,453,283; 5,443,846; 5,403,593; 5,378,462; 5,350,584; 5,283,065; 5,273,758; 5,266,331; 5,202,128; 5,183,690; 5,178,868; 5,126,145; 5,073,379; 5,023,089; 5,007,790; 4,970,075; 4,959,208; 4,59,208; 4,861,598; 4,844,909; 4,834,984; 4,828,836; 4,806,337; 4,801,460; 4,764,378; 4,421,736; 4,344,431; 4,343,789; 4,346,709; 4,230,687; 4,132,753; 5,591,452; 5,965,161; 5,958,452; 6,254,887; 6,156,342; 5,395,626; 5,474,786; and 5,919,826.

In a further exemplary embodiment, the tablet is characterized as an osmotic device for the controlled delivery of the active agent to an environment of use. Exemplary osmotic devices include those disclosed in the following patents, each of which is incorporated by reference.

U.S. Pat. No. 4,014,334 to Theeuwes et al., (“the '334 Patent”) which discloses an osmotic device for the controlled and continuous delivery of a drug wherein the device comprises: a) a core containing a drug and an osmotic agent; b) a semipermeable laminate, surrounding the core, which includes an external semipermeable lamina and an internal semipermeable lamina; and c) a passageway which communicates the core with the exterior of the device. The two semipermeable laminae maintain their chemical and physical integrity in the presence of the drug and fluid from the environment. The passageway disclosed in the '334 Patent includes an aperture, orifice or bore through the laminate formed by mechanical procedures, or by eroding an erodible element, such as a gelatin plug, in the environment of use.

U.S. Pat. No. 4,576,604 to Guittard et al. (“the '604 Patent”) discloses several different embodiments of an osmotic device having a drug in the core and at least one lamina surrounding the core. Specifically, one embodiment of the osmotic device comprises: a) a core containing a drug formulation which can include an osmotic agent for controlled release of the drug; b) a semipermeable wall comprising an inner semipermeable lamina, a middle microporous lamina, and an outer water soluble lamina containing drug; and c) a passageway which communicates the core with the exterior of the device.

U.S. Pat. No. 4,673,405 to Guittard et al. (“the '405 Patent”) discloses an osmotic device comprising: a) a core, or compartment, containing a beneficial agent; b) an inert semipermeable wall containing a beneficial agent surrounding the core; and c) at least one passageway in the wall of the osmotic device which is formed when the osmotic device is in the fluid environment of use and the fluid contacts and thus releases the beneficial agent in the wall, wherein the formed passageway communicates with the compartment in the osmotic device and the exterior of the device for dispersing the beneficial agent from the compartment when the device is in the fluid environment of use. The '405 Patent discloses the use of an erodible element to form the passageway.

U.S. Pat. No. 5,558,879 to Chen et al. (“the '879 Patent”) discloses a controlled release tablet for water-soluble drugs in which a passageway is formed in the environment of use, i.e., the GI tract of a person receiving the formulation. Specifically, the controlled release tablet consists essentially of: a) a core containing a drug, 5-20% by weight of a water soluble osmotic agent, a water soluble polymer binder and a pharmaceutical carrier; and b) a dual layer membrane coating around the core consisting essentially of: (1) an inner sustained release coating containing a plasticized water insoluble polymer and a water soluble polymer; and (2) an outer immediate release coating containing a drug and a water soluble polymer.

U.S. Pat. No. 4,810,502 to Ayer et al. (“the '502 Patent”) discloses an osmotic dosage form for delivering a single drug or a combination of active drugs which comprises: a) a core containing the first and second drugs; b) a wall surrounding the core comprising cellulose acylate and hydroxypropylcellulose; c) a passageway in the wall for delivering the drug(s); and d) a lamina on the outside of the wall comprising the active drug(s), at least one of hydroxypropylcellulose and hydroxypropyl methylcellulose, and poly(ethylene oxide) for enhancing the mechanical integrity and pharmacokinetics of the wall.

U.S. Pat. No. 4,801,461 to Hamel et al. (“the '461 Patent”) discloses an osmotic dosage form for delivering an active drug. Specifically, the osmotic dosage form comprises: a) a core containing varying amounts of the active drug; b) a semipermeable wall surrounding the core comprising varying amounts of cellulose acetate or cellulose triacetate and varying amounts of hydroxypropylcellulose; c) a passageway in the wall for delivering the drug from the core; and optionally d) a lamina on the outside of the wall comprising the active drug. The core can also contain one or more of sodium chloride, microcrystalline cellulose, hydroxypropyl methylcellulose, magnesium stearate, and poly(vinylpyrrolidone). The passageway of this device can extend through the semipermeable wall alone or through both the semipermeable wall and the outer lamina. The passageway also includes materials that erode or leach in the environment of use.

U.S. Pat. No. 5,681,584 to Savastano et al. (“the '584 Patent”) discloses a controlled release drug delivery device comprising: a) a core containing a drug, an optional osmotic agent and optional excipients; b) a delayed release jacket comprising at least one of a binder, an osmotic agent and a lubricant surrounding the core; c) a semipermeable membrane surrounding the delayed release jacket and optionally having a passageway; d) a drug-containing layer either on the outside of the semipermeable membrane or between the semipermeable membrane and the delayed release jacket; and e) an optional enteric coat either on the outside of the drug-containing layer, between the drug-containing layer and the semipermeable membrane or on the outside of the semipermeable membrane when the drug-containing layer is between the delayed release jacket and the semipermeable membrane.

U.S. Pat. No. 6,004,584 to Faour et al. (“the Faour '584 Patent”) discloses an osmotic device capable of providing a broader range of independent release profiles for one or more active agents either simultaneously or sequentially due to the particular improvements. The device includes a compressed core comprising a first active agent and an osmotic agent for controlled and continuous release of the drug; b) a semipermeable membrane surrounding the core and having a preformed passageway therein, the membrane being permeable to a fluid in the environment of use and substantially impermeable to the first active agent; c) an inert, completely erodible water soluble polymer coat comprising poly(vinylpyrrolidone)-(vinyl acetate) copolymer partially or substantially completely surrounding the semipermeable membrane and plugging the passageway in the wall; and d) an external coat comprising a second active agent for immediate release of the drug, wherein the first active agent is released from the core after the polymer coat has partially or completely dissolved or eroded, and the first and second active agents are released into the same or different environments of use to provide a controlled delivery of the one or more active agent. The Faour '584 Patent teaches that the first and second active drug may be the same drug.

Pharmaceutical compositions according to the invention may also comprise one or more binding agents, filling agents, lubricating agents, suspending agents, sweeteners, flavoring agents, preservatives, buffers, wetting agents, disintegrants, effervescent agents, and other excipients. Such excipients are known in the art.

Examples of filling agents are lactose monohydrate, lactose anhydrous, and various starches; examples of binding agents are various celluloses and cross-linked polyvinylpyrrolidone, microcrystalline cellulose, such as Avicel® PH101 and Avicel® PH102, microcrystalline cellulose, and silicified microcrystalline cellulose (ProSolv SMCC™).

Suitable lubricants, including agents that act on the flowability of the powder to be compressed, are colloidal silicon dioxide, such as Aerosil® 200, talc, stearic acid, magnesium stearate, calcium stearate, and silica gel.

Examples of sweeteners are any natural or artificial sweetener, such as sucrose, xylitol, sodium saccharin, cyclamate, aspartame, and acsulfame. Examples of flavoring agents are Magnasweet® (trademark of MAFCO), bubble gum flavor, and fruit flavors, and the like.

Examples of preservatives are potassium sorbate, methylparaben, propylparaben, benzoic acid and its salts, other esters of parahydroxybenzoic acid such as butylparaben, alcohols such as ethyl or benzyl alcohol, phenolic compounds such as phenol, or quartemary compounds such as benzalkonium chloride.

Suitable diluents include pharmaceutically acceptable inert fillers, such as microcrystalline cellulose, lactose, dibasic calcium phosphate, saccharides, and/or mixtures of any of the foregoing. Examples of diluents include microcrystalline cellulose, such as Avicel® PH101 and Avicel® PH102; lactose such as lactose monohydrate, lactose anhydrous, and Pharmatosee DCL21; dibasic calcium phosphate such as Emcompress®; mannitol; starch; sorbitol; sucrose; and glucose.

Suitable disintegrants include lightly crosslinked polyvinyl pyrrolidone, corn starch, potato starch, maize starch, and modified starches, croscarmellose sodium, cross-povidone, sodium starch glycolate, and mixtures thereof.

Examples of effervescent agents are effervescent couples such as an organic acid and a carbonate or bicarbonate. Suitable organic acids include, for example, citric, tartaric, malic, fumaric, adipic, succinic, and alginic acids and anhydrides and acid salts. Suitable carbonates and bicarbonates include, for example, sodium carbonate, sodium bicarbonate, potassium carbonate, potassium bicarbonate, magnesium carbonate, sodium glycine carbonate, L-lysine carbonate, and arginine carbonate. Alternatively, only the sodium bicarbonate component of the effervescent couple may be present.

In another aspect of the invention the imatinib compound is present in a nanoparticulate form. Non-limiting discussion of nanoparticulate form of imatinib mesylate is provided in U.S. Publication 20060275372, which is incorporated herein by reference in its entirety. Briefly, the nanoparticulate form of imatinib mesylate includes stable imatinib mesylate particles with an effective average particle size of less than about 2000 nm. Preferably, the effective average particle size is less than about 1900 nm, less than about 1800 nm, less than about 1700 nm, less than about 1600 nm, less than about 1500 nm, less than about 1400 nm, less than about 1300 nm, less than about 1200 nm, less than about 1100 nm, less than about 1000 nm, less than about 900 run, less than about 800 nm, less than about 700 nm, less than about 650 nm, less than about 600 nm, less than about 550 nm, less than about 500 nm, less than about 450, less than about 400 nm, less than about 350 nm, less than about 300 nm, less than about 250 nm, less than about 200 nm, less than about 150 nm, less than about 100 nm, less than about 75 nm, or less than about 50 nm, as measured by light-scattering methods, microscopy, or other appropriate methods. Such methods suitable for measuring effective average particle size are known to a person of ordinary skill in the art.

The nanoparticles of the imatinib compound also comprise at least one surface stabilizer. The stabilizers may act to stabilize the active agent particles at a desired particle size when the active agent particles precipitate out of solution when exposed to a neutral pH environment.

Suitable surface stabilizers include hydroxypropyl methylcellulose (now known as hypromellose), hydroxypropylcellulose, polyvinylpyrrolidone, sodium lauryl sulfate, dioctylsulfosuccinate (dioctyl sodium sulfosuccinate), gelatin, casein, lecithin (phosphatides), dextran, gum acacia, cholesterol, tragacanth, stearic acid, benzalkonium chloride, calcium stearate, glycerol monostearate, cetostearyl alcohol, cetomacrogol emulsifying wax, sorbitan esters, polyoxyethylene alkyl ethers (e.g., macrogol ethers such as cetomacrogol 1000), polyoxyethylene castor oil derivatives, polyoxyethylene sorbitan fatty acid esters (e.g., the commercially available Tweens® such as e.g., Tween® 20 and Tween® 80 (ICI Specialty Chemicals)); polyethylene glycols (e.g., Carbowaxs® 3550 and 934 (Union Carbide)), polyoxyethylene stearates, colloidal silicon dioxide, phosphates, carboxymethylcellulose calcium, carboxymethylcellulose sodium, methylcellulose, hydroxyethylcellulose, hypromellose phthalate, noncrystalline cellulose, magnesium aluminium silicate, triethanolamine, polyvinyl alcohol (PVA), 4-(1,1,3,3-tetramethylbutyl)-phenol polymer with ethylene oxide and formaldehyde (also known as tyloxapol, superione, and triton), poloxamers (e.g., Pluronics® F68 and F108, which are block copolymers of ethylene oxide and propylene oxide); poloxamines (e.g., Tetronic® 908, also known as Poloxamine™ 908, which is a tetrafunctional block copolymer derived from sequential addition of propylene oxide and ethylene oxide to ethylenediamine (BASF Wyandotte Corporation, Parsippany, N.J.)); Tetronic® 1508 (T-1508) (BASF Wyandotte Corporation), Tritons® X-200, which is an alkyl aryl polyether sulfonate (Rohm and Haas); Crodestas™ F-110, which is a mixture of sucrose stearate and sucrose distearate (Croda Inc.); p-isononylphenoxypoly-(glycidol), also known as Olin®-1OG or Surfactant™ 10-G (Olin Chemicals, Stamford, Conn.); Crodestas™ SL-40 (Croda, Inc.); and SA9OHCO, which is C₁₈H₃₇CH₂(CON(CH₃)—CH₂(CHOH)₄(CH₂0H)₂ (Eastman Kodak Co.); decanoyl-N-methylglucamide; n-decyl β-D-glucopyranoside; n-decyl β-D-maltopyranoside; n-dodecyl β-D-glucopyranoside; n-dodecyl β-D-maltoside; heptanoyl-N-methylglucamide; n-heptyl-β-D-glucopyranoside; n-heptyl β-D-thioglucoside; n-hexyl β-D-glucopyranoside; nonanoyl-N-methylglucamide; n-noyl β-D-glucopyranoside; octanoyl-N-methylglucamide; n-octyl-β-D-glucopyranoside; octyl β-D-thioglucopyranoside; PEG-phospholipid, PEG-cholesterol, PEG-cholesterol derivative, PEG-vitamin A, PEG-vitamin E, lysozyme, random copolymers of vinyl pyrrolidone and vinyl acetate, and the like.

Examples of useful cationic surface stabilizers include, but are not limited to, polymers, biopolymers, polysaccharides, cellulosics, alginates, phospholipids, and nonpolymeric compounds, such as zwitterionic stabilizers, poly-n-methylpyridinium, anthryul pyridinium chloride, cationic phospholipids, chitosan, polylysine, polyvinylimidazole, polybrene, polymethylmethacrylate trimethylammoniumbromide bromide (PMMTMABr), hexyldesyltrimethylammonium bromide (HDMAB), and polyvinylpyrrolidone-2-dimethylaminoethyl methacrylate dimethyl sulfate.

Other useful cationic stabilizers include, but are not limited to, cationic lipids, sulfonium, phosphonium, and quarternary ammonium compounds, such as stearyltrimethylammonium chloride, benzyl-di(2-chloroethyl)ethylammonium bromide, coconut trimethyl ammonium chloride or bromide, coconut methyl dihydroxyethyl ammonium chloride or bromide, decyl triethyl ammonium chloride, decyl dimethyl hydroxyethyl ammonium chloride or bromide, C₁₂₋₁₅dimethyl hydroxyethyl ammonium chloride or bromide, coconut dimethyl hydroxyethyl ammonium chloride or bromide, myristyl trimethyl ammonium methyl sulphate, lauryl dimethyl benzyl ammonium chloride or bromide, lauryl dimethyl (ethenoxy)₄ ammonium chloride or bromide, N-alkyl (C₁₂₋₁₈)dimethylbenzyl ammonium chloride, N-alkyl (C₁₄₋₁₈)dimethyl-benzyl ammonium chloride, N-tetradecylidmethylbenzyl ammonium chloride monohydrate, dimethyl didecyl ammonium chloride, N-alkyl and (C₁₂₋₁₄) dimethyl 1-napthylmethyl ammonium chloride, trimethylammonium halide, alkyl-trimethylammonium salts and dialkyl-dimethylammonium salts, lauryl trimethyl ammonium chloride, ethoxylated alkyamidoalkyldialkylammonium salt and/or an ethoxylated trialkyl ammonium salt, dialkylbenzene dialkylammonium chloride, N-didecyldimethyl ammonium chloride, N-tetradecyldimethylbenzyl ammonium, chloride monohydrate, N-alkyl(C₁₂₋₁₄) dimethyl 1-naphthylmethyl ammonium chloride and dodecyldimethylbenzyl ammonium chloride, dialkyl benzenealkyl ammonium chloride, lauryl trimethyl ammonium chloride, alkylbenzyl methyl ammonium chloride, alkyl benzyl dimethyl ammonium bromide, C₁₂, C₁₅, C₁₇ trimethyl ammonium bromides, dodecylbenzyl triethyl ammonium chloride, poly-diallyldimethylammonium chloride (DADMAC), dimethyl ammonium chlorides, alkyldimethylammonium halogenides, tricetyl methyl ammonium chloride, decyltrimethylammonium bromide, dodecyltriethylammonium bromide, tetradecyltrimethylammonium bromide, methyl trioctylammonium chloride (ALIQUAT 336™), POLYQUAT 10™, tetrabutylammonium bromide, benzyl trimethylammonium bromide, choline esters (such as choline esters of fatty acids), benzalkonium chloride, stearalkonium chloride compounds (such as stearyltrimonium chloride and Di-stearyldimonium chloride), cetyl pyridinium bromide or chloride, halide salts of quaternized polyoxyethylalkylamines, MIRAPOL™ and ALKAQUAT™ (Alkaril Chemical Company), alkyl pyridinium salts; amines, such as alkylamines, dialkylamines, alkanolamines, polyethylenepolyamines, N,N-dialkylaminoalkyl acrylates, and vinyl pyridine, amine salts, such as lauryl amine acetate, stearyl amine acetate, alkylpyridinium salt, and alkylimidazolium salt, and amine oxides; imide azolinium salts; protonated quaternary acrylamides; methylated quaternary polymers, such as poly[diallyl dimethylammonium chloride] and poly-[N-methyl vinyl pyridinium chloride]; and cationic guar.

Such exemplary cationic surface stabilizers and other useful cationic surface stabilizers are described in J. Cross and E. Singer, Cationic Surfactants: Analytical and Biological Evaluation (Marcel Dekker, 1994); P. and D. Rubingh (Editor), Cationic Surfactants: Physical Chemistry (Marcel Dekker, 1991); and J. Richmond, Cationic Surfactants: Organic Chemistry, (Marcel Dekker, 1990).

Nonpolymeric surface stabilizers are any nonpolymeric compound, such benzalkonium chloride, a carbonium compound, a phosphonium compound, an oxonium compound, a halonium compound, a cationic organometallic compound, a quarternary phosphorous compound, a pyridinium compound, an anilinium compound, an ammonium compound, a hydroxylammonium compound, a primary ammonium compound, a secondary ammonium compound, a tertiary ammonium compound, and quarternary ammonium compounds of the formula NR₁R₂R₃R₄⁽⁺⁾. For compounds of the formula NR₁R₂R₃R₄⁽⁺⁾:

(i) none of R₁-R₄ are CH₃;

(ii) one of R₁-R₄ is CH₃;

(iii) three of R₁-R₄ are CH₃;

(iv) all of R₁-R₄ are CH₃;

(v) two of R₁-R₄ are CH₃, one of R₁-R₄ is C₆H₅CH₂, and one of R₁-R₄ is an alkyl chain of seven carbon atoms or less;

(vi) two of R₁-R₄ are CH₃, one of R₁-R₄ is C₆H₅CH₂, and one of R₁-R₄ is an alkyl chain of nineteen carbon atoms or more;

(vii) two of R₁-R₄ are CH₃ and one of R₁-R₄ is the group C₆H₅(CH₂)ₙ, where n>1;

(viii) two of R₁-R₄ are CH₃, one of R₁-R₄ is C₆H₅CH₂, and one of R₁-R₄ comprises at least one heteroatom;

(ix) two of R₁-R₄ are CH₃, one of R₁-R₄ is C₆H₅CH₂, and one of R₁-R₄ comprises at least one halogen;

(x) two of R₁-R₄ are CH₃, one of R₁-R₄ is C₆H₅CH₂, and one of R₁-R₄ comprises at least one cyclic fragment;

(xi) two of R₁-R₄ are CH₃ and one of R₁-R₄ is a phenyl ring; or

(xii) two of R₁-R₄ are CH₃ and two of R₁-R₄ are purely aliphatic fragments.

Such compounds include, but are not limited to, behenalkonium chloride, benzethonium chloride, cetylpyridinium chloride, behentrimonium chloride, lauralkonium chloride, cetalkonium chloride, cetrimonium bromide, cetrimonium chloride, cethylamine hydrofluoride, chlorallylmethenamine chloride (Quaternium-15), distearyldimonium chloride (Quaternium-5), dodecyl dimethyl ethylbenzyl ammonium chloride(Quaternium-14), Quaternium-22, Quaternium-26, Quaternium-18 hectorite, dimethylaminoethylchloride hydrochloride, cysteine hydrochloride, diethanolammonium POE (10) oletyl ether phosphate, diethanolammonium POE (3)oleyl ether phosphate, tallow alkonium chloride, dimethyl dioctadecylammoniumbentonite, stearalkonium chloride, domiphen bromide, denatonium benzoate, myristalkonium chloride, laurtrimonium chloride, ethylenediamine dihydrochloride, guanidine hydrochloride, pyridoxine HCl, iofetamine hydrochloride, meglumine hydrochloride, methylbenzethonium chloride, myrtrimonium bromide, oleyltrimonium chloride, polyquaternium-1, procainehydrochloride, cocobetaine, stearalkonium bentonite, stearalkoniumhectonite, stearyl trihydroxyethyl propylenediamine dihydrofluoride, tallowtrimonium chloride, and hexadecyltrimethyl ammonium bromide.

Many surface stabilizers are commercially available and/or can be prepared by techniques known in the art. See e.g., Handbook of Pharmaceutical Excipients, published jointly by the American Pharmaceutical Association and The Pharmaceutical Society of Great Britain (The Pharmaceutical Press, 2000), specifically incorporated by reference.

The surface stabilizers are commercially available and/or can be prepared by techniques known in the art. Most of these surface stabilizers are known pharmaceutical excipients and are described in detail in the Handbook of Pharmaceutical Excipients, published jointly by the American Pharmaceutical Association and The Pharmaceutical Society of Great Britain (The Pharmaceutical Press, 2000), specifically incorporated by reference.

The imatinib compound and surface stabilizer may be present in the pharmaceutical compositions disclosed herein at any suitable ratio (w/w). For example, in some embodiments the pharmaceutical compositions include the imatinib mesylate composition and the surface stabilizer at a ratio of about 20:1, 15:1, 10:1, 8:1, 7:1, 6:1, 5:1, 4:1, 3:1, 2:1 (w/w), or any range defined by said ratios (for example, but not limited to about 20:1-2:1, about 10:1-4:1, and about 8:1-5:1).

The relative amounts of the imatinib compound and one or more surface stabilizers can vary widely. The optimal amount of the individual components can depend, for example, upon the particular imatinib mesylate selected, the hydrophilic lipophilic balance (HLB), melting point, and the surface tension of water solutions of the stabilizer, etc.

The concentration of the imatinib mesylate can vary from about 99.5% to about 0.001%, from about 95% to about 0.1%, or from about 90% to about 0.5%, by weight, based on the total combined dry weight of the imatinib mesylate and at least one surface stabilizer, not including other excipients.

The concentration of the at least one surface stabilizer can vary from about 0.5% to about 99.999%, from about 5.0% to about 99.9%, or from about 10% to about 99.5%, by weight, based on the total combined dry weight of the imatinib mesylate and at least one surface stabilizer, not including other excipients.

The nanoparticulate imatinib mesylate, or a salt or derivative thereof, compositions can be made using, for example, milling, homogenization, precipitation, cryogenic, or template emulsion techniques. Exemplary methods of making nanoparticulate active agent compositions are described in the '684 patent. Methods of making nanoparticulate active agent compositions are also described in U.S. Pat. No. 5,518,187 for “Method of Grinding Pharmaceutical Substances;” U.S. Pat. No. 5,718,388 for “Continuous Method of Grinding Pharmaceutical Substances;” U.S. Pat. No. 5,862,999 for “Method of Grinding Pharmaceutical Substances;” U.S. Pat. No. 5,665,331 for “Co-Microprecipitation of Nanoparticulate Pharmaceutical Agents with Crystal Growth Modifiers;” U.S. Pat. No. 5,662,883 for “Co-Microprecipitation of Nanoparticulate Pharmaceutical Agents with Crystal Growth Modifiers;” U.S. Pat. No. 5,560,932 for “Microprecipitation of Nanoparticulate Pharmaceutical Agents;” U.S. Pat. No. 5,543,133 for “Process of Preparing X-Ray Contrast Compositions Containing Nanoparticles;” U.S. Pat. No. 5,534,270 for “Method of Preparing Stable Drug Nanoparticles;” U.S. Pat. No. 5,510,118 for “Process of Preparing Therapeutic Compositions Containing Nanoparticles;” and U.S. Pat. No. 5,470,583 for “Method of Preparing Nanoparticle Compositions Containing Charged Phospholipids to Reduce Aggregation,” all of which are specifically incorporated by reference. For a more detailed discussion of methods for preparing the nanoparticulate compositions of imatinib compounds, see US 20060275372.

The nanoparticulate form of the imatinib compounds provides multiple advantages compared to conventional (i.e., non-nanoparticulate) formulations of imatinib. Such advantages include, without limitations, increased redispersibility due to the fact that stable nanoparticles of imatinib do not agglomerate, improved pharmacokinetics properties, including increased Cₘₐₓ (maximal plasma concentration), increased AUC (area under the curve), and decreased Tₘₐₓ.

Further, the administration of the nanoparticulate imatinib compound formulation to a subject in a fasted state is bioequivalent to administration of the composition to a subject in a fed state.

In addition, the compositions of the instant invention may optionally comprise at least a second active ingredient, which may optionally be present in a nanoparticulate form. Generally, the second active ingredient will potentiate the anti-cancer effect of imatinib and/or minimize the side effects of the imatinib compound. Thus, in different exemplary embodiments, compounds suitable as at least the second active ingredient include anti-emetic compounds, anti-diarrhea compounds, and H₂ antagonists.

Notably, since the coating of Gleevec® tables comprises iron oxide, concerns exist that certain treatment regimens may cause iron overload in the patient. For example, the the official website of Gleevec® (http://www.gleevec.com) advises the patients to tell his or her doctor if the patient is taking or plans to take iron supplements. Further, the website discloses that patients who ingest 800 mg (or more) daily, should use two 400 mg tablets to lower their iron exposure. Accordingly, another embodiment of the invention provides a composition which has an equivalent of 800 mg of imatinib and a non-toxic amount of iron.

Although the invention herein has been described with reference to particular embodiments, it is to be understood that these embodiments are merely illustrative of the principles and applications of the present invention. It is therefore to be understood that numerous modifications may be made to the illustrative embodiments and that other arrangements may be devised without departing from the spirit and scope of the present invention as defined by the following claims.

All publications cited in the specification, both patent publications and non-patent publications, are indicative of the level of skill of those skilled in the art to which this invention pertains. All these publications are herein fully incorporated by reference to the same extent as if each individual publication were specifically and individually indicated as being incorporated by reference.

Also, unless indicated to the contrary, where various numerical values are provided for embodiments, additional embodiments are described by taking any 2 different values as the endpoints of a range. Such ranges are also within the scope of the described invention.

What is claimed is:

1. An oral formulation for administering to a subject comprising
a) an imatinib compound; and
b) an enteric matrix or enteric coating or a combination thereof;
whereby at least 80% of the imatinib compound is released in the small intestine of the subject.

2. The formulation of claim 1, wherein the imatinib compound is imatinib mesylate.

3. The formulation of claim 1, wherein the enteric coating is selected from cellulose acetate phthalate, cellulose acetate trimaletate, hydroxy propyl methylcellulose phthalate, polyvinyl acetate phthalate, ammonio methacrylate copolymers, poly acrylic acid and poly acrylate and methacrylate copolymers, polyvinyl acetaldiethylamino acetate, hydroxypropyl methylcellulose acetate succinate, shellac, hydrogels and gel-forming materials, carboxyvinyl polymers, sodium alginate, sodium carmellose, calcium carmellose, sodium carboxymethyl starch, poly vinyl alcohol, hydroxyethyl cellulose, methyl cellulose, gelatin, starch, hydroxypropyl cellulose, hydroxypropyl methylcellulose, polyvinylpyrrolidone, crosslinked starch, microcrystalline cellulose, chitin, aminoacryl-methacrylate copolymer, pullulan, collagen, casein, agar, gum arabic, sodium carboxymethyl cellulose, (swellable hydrophilic polymers) poly(hydroxyalkyl methacrylate) (m. wt. about 5 k-5,000 k), polyvinylpyrrolidone (m. wt. ˜10 k-360 k), anionic and cationic hydrogels, polyvinyl alcohol having a low acetate residual, a swellable mixture of agar and carboxymethyl cellulose, copolymers of maleic anhydride and styrene, ethylene, propylene or isobutylene, pectin (m. wt. ˜30 k-300 k), agar, acacia, karaya, tragacanth, algins and guar, polyacrylamides, POLYOX®, polyethylene oxides (m. wt. ˜100 k-5,000 k), AQUAKEEP® acrylate polymers, diesters of polyglucan, crosslinked polyvinyl alcohol and poly N-vinyl-2-pyrrolidone, sodium starch glucolate, polysaccharides, methyl cellulose, sodium or calcium carboxymethyl cellulose, hydroxypropyl methyl cellulose, hydroxypropyl cellulose, hydroxyethyl cellulose, nitro cellulose, carboxymethyl cellulose, cellulose ethers, polyethylene oxides, methyl ethyl cellulose, ethylhydroxy ethylcellulose, cellulose acetate, cellulose butyrate, cellulose propionate, gelatin, collagen, starch, maltodextin, pullulan, polyvinyl pyrrolidone, polyvinyl alcohol, polyvinyl acetate, glycerol fatty acid esters, polyacrylamide, polyacrylic acid, copolymers of methacrylic acid or methacrylic acid, sorbitan esters, natural gums, lecithins, pectin, alginates, ammonia alginate, sodium, calcium, potassium alginates, propylene glycol alginate, agar, arabic, karaya, locust bean, tragacanth, carrageens, guar, xanthan, scleroglucan and mixtures and blends thereof and any combination thereof.

4. The formulation of claim 1, whereby at least 85 % of the imatinib compound is released in the small intestine of the subject.

5. The formulation of claim 4, whereby at least 90% of the imatinib compound is released in the small intestine of the subject.

6. The formulation of claim 5, whereby at least 95% of the imatinib compound is released in the small intestine of the subject.

7. The formulation of claim 6, whereby at least 99% of the imatinib compound is released in the small intestine of the subject.

8. The formulation of claim 1, wherein at least a portion of the imatinib compound is in a nanoparticulate form, and wherein the nanoparticles of the imatinib compound further comprise at least one surface stabilizer.

9. The formulation of claim 8, wherein the at least one surface stabilizer is selected from the group consisting of cetyl pyridinium chloride, gelatin, casein, phosphatides, dextran, glycerol, gum acacia, cholesterol, tragacanth, stearic acid, benzalkonium chloride, calcium stearate, glycerol monostearate, cetostearyl alcohol, cetomacrogol emulsifying wax, sorbitan esters, polyoxyethylene alkyl ethers, polyoxyethylene castor oil derivatives, polyoxyethylene sorbitan fatty acid esters, polyethylene glycols, dodecyl trimethyl ammonium bromide, polyoxyethylene stearates, colloidal silicon dioxide, phosphates, sodium dodecylsulfate, carboxymethylcellulose calcium, hydroxypropyl celluloses, hypromellose, carboxymethylcellulose sodium, methylcellulose, hydroxyethylcellulose, hypromellose phthalate, noncrystalline cellulose, magnesium aluminum silicate, triethanolamine, polyvinyl alcohol, polyvinylpyrrolidone, 4-(1,1,3,3-tetramethylbutyl)-phenol polymer with ethylene oxide and formaldehyde, poloxamers; poloxamines, a charged phospholipid, dioctylsulfosuccinate, dialkylesters of sodium sulfosuccinic acid, sodium lauryl sulfate, alkyl aryl polyether sulfonates, mixtures of sucrose stearate and sucrose distearate, p-isononylphenoxypoly-(glycidol), decanoyl-N-methylglucamide; n-decyl β-D-glucopyranoside; n-decyl β-D-maltopyranoside; n-dodecyl β-D-glucopyranoside; n-dodecyl β-D-maltoside; heptanoyl-N-methylglucamide; n-heptyl-β-D-glucopyranoside; n-heptyl β-D-thioglucoside; n-hexyl β-D-glucopyranoside; nonanoyl-N-methylglucamide; n-noyl ≠-D-glucopyranoside; octanoyl-N-methylglucamide; n-octyl-β-D-glucopyranoside; octyl β-D-thioglucopyranoside; lysozyme, PEG-phospholipid, PEG-cholesterol, PEG-cholesterol derivative, PEG-vitamin A, PEG-vitamin E, random copolymers of vinyl acetate and vinyl pyrrolidone, a cationic polymer, a cationic biopolymer, a cationic polysaccharide, a cationic cellulosic, a cationic alginate, a cationic nonpolymeric compound, a cationic phospholipids, cationic lipids, polymethylmethacrylate trimethylammonium bromide, sulfonium compounds, polyvinylpyrrolidone-2-dimethylaminoethyl methacrylate dimethyl sulfate, hexadecyltrimethyl ammonium bromide, phosphonium compounds, quarternary ammonium compounds, benzyl-di(2-chloroethyl)ethylammonium bromide, coconut trimethyl ammonium chloride, coconut trimethyl ammonium bromide, coconut methyl dihydroxyethyl ammonium chloride, coconut methyl dihydroxyethyl ammonium bromide, decyl triethyl ammonium chloride, decyl dimethyl hydroxyethyl ammonium chloride, decyl dimethyl hydroxyethyl ammonium chloride bromide, C₁₂₋₁₅ dimethyl hydroxyethyl ammonium chloride, C₁₂₋₁₅ dimethyl hydroxyethyl ammonium chloride bromide, coconut dimethyl hydroxyethyl ammonium chloride, coconut dimethyl hydroxyethyl ammonium bromide, myristyl trimethyl ammonium methyl sulphate, lauryl dimethyl benzyl ammonium chloride, lauryl dimethyl benzyl ammonium bromide, lauryl dimethyl (ethenoxy)₄ ammonium chloride, lauryl dimethyl (ethenoxy)₄ ammonium bromide, N-alkyl (C₁₂₋₁₈)dimethylbenzyl ammonium chloride, N-alkyl (C₁₂₋₁₄)dimethyl-benzyl ammonium chloride, N-tetradecylidmethylbenzyl ammonium chloride monohydrate, dimethyl didecyl ammonium chloride, N-alkyl and (C₁₂₋₁₄) dimethyl 1-napthylmethyl ammonium chloride, trimethylammonium halide, alkyl-trimethylammonium salts, dialkyl-dimethylammonium salts, lauryl trimethyl ammonium chloride, ethoxylated alkyamidoalkyldialkylammonium salt, an ethoxylated trialkyl ammonium salt, dialkylbenzene dialkylammonium chloride, N-didecyldimethyl ammonium chloride, N-tetradecyldimethylbenzyl ammonium, chloride monohydrate, N-alkyl (C₁₂₋₁₄) dimethyl 1-naphthylmethyl ammonium chloride, dodecyldimethylbenzyl ammonium chloride, dialkyl benzenealkyl ammonium chloride, lauryl trimethyl ammonium chloride, alkylbenzyl methyl ammonium chloride, alkyl benzyl dimethyl ammonium bromide, C₁₂ trimethyl ammonium bromides, C₁₅ trimethyl ammonium bromides, C₁₇ trimethyl ammonium bromides, dodecylbenzyl triethyl ammonium chloride, poly-diallyldimethylammonium chloride, dimethyl ammonium chlorides, alkyldimethylammonium halogenides, tricetyl methyl ammonium chloride, decyltrimethylammonium bromide, dodecyltriethylammonium bromide, tetradecyltrimethylammonium bromide, methyl trioctylammonium chloride, tetrabutylammonium bromide, benzyl trimethylammonium bromide, choline esters, benzalkonium chloride, stearalkonium chloride compounds, cetyl pyridinium bromide, cetyl pyridinium chloride, halide salts of quatemized polyoxyethylalkylamines, alkyl pyridinium salts; amines, amine salts, amine oxides, imide azolinium salts, protonated quaternary acrylamides, methylated quaternary polymers, and cationic guar.

10. The formulation of claim 8, wherein the nanoparticles have an average diameter of less than about 2000 nm.

11. The formulation of claim 1, comprising a first population of imatinib compound-containing particles and at least one subsequent population of active ingredient-containing particles, wherein the subsequent population of at least a second active ingredient-containing particles further comprises a modified release coating or, alternatively or additionally, a modified release matrix material, such that the imatinib compound and at least the second active ingredient reach their respective peak plasma concentrations in a pre-determined time interval.

12. The formulation of claim 11, wherein at least the second active ingredient is not the imatinib compound.

13. The formulation of claim 12, wherein at least the second active ingredient is selected from anti-emetic compounds, anti-diarrhea compounds, and H₂ antagonists.

14. The formulation of claim 11, wherein members of the first and the subsequent populations of particles each have a diameter of less than approximately 2000 nm.

15. The formulation of claim 1, wherein the imatinib compound is present in the amount equivalent to at least about 400 mg of imatinib.

16. The formulation of claim 15, wherein the imatinib compound is present in the amount equivalent to at least about 600 mg of imatinib.

17. The formulation of claim 16, wherein the imatinib compound is present in the amount equivalent to at least about 800 mg of imatinib.

18. The formulation of claim 1, further comprising a non-toxic amount of iron.

19. A method of treating a subject having a disease amenable to imatinib therapy, comprising administering to a subject the formulation of claim 1.

20. The method of claim 19, wherein a single daily dose of the formulation comprises the imatinib compound in the amount equivalent to about 800 mg of imatinib.
